(19) United States
(12) Patent Application Publication
(10) Pub. No.: US 2009/0062386 A1
(43) Pub. Date: Mar. 5, 2009

(54) Simulants of Toxants for Training and Testing

(75) Inventors: Charles W. Martin, Springfield, IL (US); Joan A. Stader, Ballwin, MO (US); Jeffry Golden, Creve Coeur, MO (US)

Correspondence Address: HUSCH BLACKWELL SANDERS LLP, 190 CARONDELET PLAZA, SUITE 600, ST. LOUIS, MO, 63105-3441, US

(73) Assignee: CLEAN EARTH TECHNOLOGIES, LLC, St. Louis, MO (US)

(21) Appl. No.: 11/847,913

(22) Filed: Aug. 30, 2007

(51) Int. Cl. A61K 31/225; C07C 69/18

(52) U.S. Cl. 514/547; 560/231

(57) ABSTRACT

Compositions are formulated with generally regarded as safe (GRAS) ingredients for use as chemical simulants of toxants such as chemical warfare agents. The compositions can be used for training exercises, testing, and research studies and they can be applied safely to human skin. They include ultraviolet-excited fluorescent ingredients that make possible visible viewing of the simulants. The chemical simulants have good fidelity with the physical properties of toxants, for example, vapor pressure, volatility, persistence, viscosity, response to oxidative, hydrolysis, and perhydrolysis decontaminants, and they can be detected by commonly used portable instruments used to detect toxants.

### STATEMENT REGARDING FEDERALLY SPONSORED RESEARCH

The development of the compositions of this invention was supported, in part, by the Technical Support Working Group, an agency of the Federal Government, under contract N411756-05-C-4778. The Federal Government retains Government Purpose Rights, which include the right to use, modify, perform, display, release, or disclose technical data in whole or in part, in any manner or for any government purpose whatsoever, and to have or authorize others to do so in the performance of a Government Contract.

### CROSS-REFERENCE TO RELATED APPLICATIONS

None

### APPENDIX

Not Applicable.

### BACKGROUND OF THE INVENTION

1. Field of the Invention

The invention relates to chemical compositions for use as simulants of toxants in training exercises, testing, and research studies that involve the detection and decontamination, especially for situations that involve direct human exposure.

### RELATED ART

Simulants of chemical toxants, such as chemical warfare agents and toxic industrial chemicals can provide emergency responders, military personnel, researchers, trainers, and training evaluators with means to learn, practice and evaluate methods and techniques for detection and decontamination in training exercises, testing, and research studies. Such simulants can also be used for research studies to determine the transport and spread of contamination onto people, from person to person, and in the environment. Further, such simulants can be incorporated into training kits with simulants for other hazardous materials such as radiological and biological materials, and explosives.

The selection of simulants for the development and evaluation of detectors and decontamination technologies generally has focused on matching simulant physical and/or chemical properties with a toxant or class of toxants. For development, testing, evaluation, and training in a laboratory or other controlled setting where containment measures are employed and personal protection equipment (PPE) are used so that human exposure is avoided, simulant toxicity is a lesser concern, and the use of a high fidelity simulant, even one with significant toxicity, in place of chemical agents or TICs is advantageous because the agents or TICs may be controlled substances and have cost, and handling and safeguard expenses that may be prohibitive or limiting. In training exercises where unprotected human exposure is avoided, for example, where participants wear adequate PPE, training with the use of toxic simulants with much lower toxicity than chemical agents can be conducted with good fidelity and realism.

Prior training kits and simulants have not adequately addressed the requirements for realistic training for detection or decontamination in which unprotected persons are exposed to and contaminated by the simulant. Further, the prior art does not provide a set of simulants based on simple compositions that have characteristics that mimic several of the physical characteristics of a corresponding set of agents. Moreover, while some of the prior art has offered simulants with characteristic odors, vapor signatures, or viscosity, none provide a set of related simulants with several of these characteristics well simulating a corresponding set of agents. Still further, for realism in training exercises, it is desirable that members of a set of simulants not be readily identifiable by odor as smelling such agents is hazardous and in an actual chemical agent event, the agents are to be identified by the use of instruments or by their physical and chemical properties, such as vapor pressure, molecular weight, viscosity, response to decontamination, or reaction with detection reagents. Further, the organophosphorous agents typically have no odor or a faint, fruity odor. Simulants often used as surrogates for such agents, for example methyl salicylate (MES), have a pungent and distinctive odor, which is not desirable for high fidelity training.

Hovanec (Registration No. H270) invented a simulant for the nerve agent VX, the simulant, which has structural similarity to the nerve agent, but being intended for use in research, development, evaluation and testing of chemically active liquid decontaminants, Hovanec's simulant is toxic and harmful to exposed humans, although it is much less toxic than the VX agent. Moreover, the simulant was not designed for use in training exercises.

Johanns, Bowen and Wyant (U.S. Pat. No. 6,027,344) disclose a simulant training kit for recognizing hazardous materials. The use of the kit was limited to visual observation of physical characteristics of the simulant to train individuals to recognize authentic contaminant. The simulants used were less hazardous than the contaminant but were not safe for use directly on humans. The simulants described by Johanns, Bowen and Wyant are not intended to be removed from their containers, while the simulants described here are intended to be distributed on a variety of surfaces including human skin.

Seitzinger and Genovese (U.S. Pat. Nos. 6,566,138 and 7,129,094) invented fluorescent simulants that comprise a vapor generating ingredient, for example MES, a fluorescent material, and a solvent for dispersion of the other ingredients, such simulants being for use in training personnel in the handling and decontamination of surfaces containing CWAs. Their simulant compositions provide a vapor signature for detection, and an odor, that uniquely identifies a given simulant, which may be an undesirable characteristic as explained above, but their simulants do not provide a vapor that mimics the volatility and vapor pressure of the corresponding specific toxants and that also provides a viscosity that corresponds to a particular agent material, and so, they teach the addition of a thickener to adjust viscosity. Moreover, their simulants do not have solubility that has good fidelity to specific toxants. Still further, they do not prescribe ingredients or fluorescent taggants that are Generally Regarded As Safe (GRAS). In this regard, they teach that the ingredients be selected so that their use does not lead to violation of the RCRA environmental protection law.

Trogen, Andersson, Berglund, and Nyholm (International Publication No. WO 03/073037 A1) invented a chemical simulant for training purposes. Their simulant compositions comprise an oil-in-water emulsion containing an organic compound such as methyl salicylate (MES) as an emulsified phase and in a proportion that is selected to ensure that the composition does not catch fire during the intended dispersal by an explosive dissemination device. Their simulant is not GRAS, does not contain a taggant, and requires dispersal as an emulsion.

Teta, Brown and Glass (International Publication No. WO 2004/040255 A2, U.S. Pat. No. 6,913,928 and U.S. Pat. No. 6,913,928) invented fluorescent chemical simulants that can be used in decontamination training operations with the prescribed method of detection being observation of visible light when illuminated by ultraviolet (UV) light. However, while their simulant composition can be adjusted to match the viscosity of specific chemical warfare agents, such simulant compositions do not possess the solubility, volatility, vapor pressure, and response to decontamination of the corresponding agents and so are of poor fidelity and realism.

The prior art has failed to provide simulant compositions comprising only GRAS ingredients, containing fluorescent taggants, and that have characteristics of good fidelity to the volatility, vapor pressure, persistence, molecular weight, relative viscosity, and response to decontamination solutions. The prior art does not describe simulants that are meant to be detected using methods and a wide variety of instruments that are commonly used by emergency responders and military personnel in training and in preparation for an actual contamination event. Further, prior art does not describe simulant compositions that are safe for use on human skin and that comprise ingredients, which are in the International Dictionary of Cosmetic Ingredients and have GRAS status.

### SUMMARY OF THE INVENTION

The simulant compositions contain mixtures of triacetin (TA) and ethyl lactate (EL) with a small admixture of curcumin or other non-toxic fluorescent taggant. The proportions of TA and EL are selected to obtain the desired physical characteristics that correspond to specific chemical toxants, for example organo-phosphorous compounds such as Tabun (GA), Sarin (GB), Soman (GD), these together being members of the G-series (G-family) of agents, the nerve agent VX, or vesicants such as sulfur mustard (HD). All of the ingredients of the simulant compositions are listed in the International Dictionary of Cosmetics and Fragrances, and they have established GRAS status. Simulant materials are non-toxic and non-irritating to human skin and mucous membranes. They produce only slight to mild ocular irritation. The simulants are environmentally safe and can be used for classroom and field operations training.

The principal ingredient, TA, is selected because it is an excellent non-toxic simulant for the nerve agent VX. TA has low vapor pressure at room temperature, and it has volatility, viscosity, appearance, molecular weight, boiling point, and solubility that are comparable to VX. By the addition of EL (even in minute quantities such that EL concentrations may vary from between greater than 0% to about 80%), these characteristics are modified so that the various members of the G-series can be simulated, either individually or by a ‘generic’ G-agent simulant. The vesicant HD and related agents are also well simulated by mixtures of TA and EL.

The TA-based simulants for chemical toxants replicate the volatility of the chemical toxants, and their dissemination results in a vapor concentration that is comparable to the toxant. This provides realistic detection conditions for a variety of toxants that span the range from low to high volatility. They are detectable by a variety of portable detectors, especially volatile organic compound detection instruments, in common use by emergency responders or military personnel. Additionally, the simulants for chemical toxants approximate the viscosity and water solubility of the chemical toxants to enable realistic training.

The fluorescent taggant makes the simulant observable by visual inspection when illuminated by UV light. Ample UV light can be obtained from a hand held UV producing lamp. In this way, persons viewing the glowing taggant can identify the presence and location of the simulant. Transport and spreading of the simulant can be determined by repeated viewing. Because of the high fidelity characteristics (e.g., volatility, viscosity, persistence, and solubility of the simulants of chemical toxants), the spread of simulant can mimic the spread of real contamination. Further, the instrument detectability and the visual detectability with UV illumination enable rapid evaluation, validation, development of tactics, techniques & procedures (TTPs), and training for detection and the use of countermeasure technologies (for example decontamination and removal). By the use of the simulant compositions of GRAS ingredients, these training, preparedness, and development activities can be performed safely, affordably, and with high fidelity to the outcomes of actual detection, avoidance, decontamination, and handling of hazardous contamination.

The invention achieves a suite of GRAS simulants for chemical toxants such as chemical warfare agents and toxic industrial chemicals, for use by emergency responders, military personnel, researchers, trainers, and training evaluators. The simulants can be assembled in a training kit that is portable and convenient to use in field exercises. The simulants of the invention can be applied to unprotected persons and animals, persons in personal protective equipment (PPE), equipment, and other objects without harm. The simulants can be detected by commonly used portable detectors such as vapor detectors, which may be used during a training exercise, and they can be detected with common laboratory instruments, too. The simulants can also be observed by UV light excited fluorescence detection, such as by a trainee, trainer or training evaluator of a training exercise.

The simulants of the present invention may be used in the development, conduct, and evaluation of training procedures and trainee proficiency evaluation related to countermeasures for chemical contaminants and in research studies of methods and techniques for the detection, sampling, and decontamination of hazardous materials after an accidental or intentional release of toxants.

The simulants of the present invention may also be used for training, research, and evaluation for detection, sampling, decontamination, and the study of the transport of chemical toxants.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 illustrates the chemical structure of (top) triacetin, (middle) ethyl lactate, (bottom) curcumin

FIG. 2 illustrates the UV-excited fluorescent chemical simulant on a human ‘victim’ during a training exercise.

FIG. 3A is a graph of the characteristics of the TA-based simulants wherein viscosity (St) is shown as a function of the EL percentage by weight.

FIG. 3B is a graph of the characteristics of the TA-based simulants wherein vapor pressure (Torr) is shown as a function of the EL percentage by weight.

FIG. 3C is a graph of the characteristics of the TA-based simulants wherein volatility (mg/m³) is shown as a function of the EL percentage by weight.

FIG. 3D is a graph of the characteristics of the TA-based simulants wherein solubility (g/g) is shown as a function of the EL percentage by weight.

FIG. 4A is a graph illustrating the vapor concentration of the chemical simulants shown as a function of temperature as measured by a commercially available MiniRae 2000® photoionization detector.

FIG. 4A is a graph illustrating the vapor concentration of the chemical simulants shown as a function of temperature as measured by a commercially available ppbRae® photoionization detector.

FIG. 5 is a graph illustrating the relative fluorescence of the chemical simulants as a function of curcumin concentration as shown for three simulant compositions: (G-s) G-agent simulant; (H-s) H-simulant; (V-s) VX simulant, wherein fluorescence is shown in FSU (fluorescein standard units).

FIG. 6A illustrates the excitation spectra of TA-based simulants with curcumin taggant, wherein the excitation wavelength was 415 nm.

FIG. 6B illustrates the emission spectra of TA-based simulants with curcumin taggant.

FIG. 7 illustrates that environmental breakdown of the chemical simulants comprises safe end products.

FIG. 8A is a bar graph of Accelerated Shelf-Life Testing of Chemical Simulants showing fluorescence activity as measured in a Victor2 plate reader, with values presented as a ratio (expressed as percent) of the signal from a fluorescein standard.

FIG. 8B is a bar graph of Accelerated Shelf-Life Testing of Chemical Simulants showing triacetin levels measured by HPLC.

FIG. 9. The chemical simulant can be applied as an aerosol spray from a common spray bottle.

### DETAILED DESCRIPTION OF THE PREFERRED EMBODIMENTS

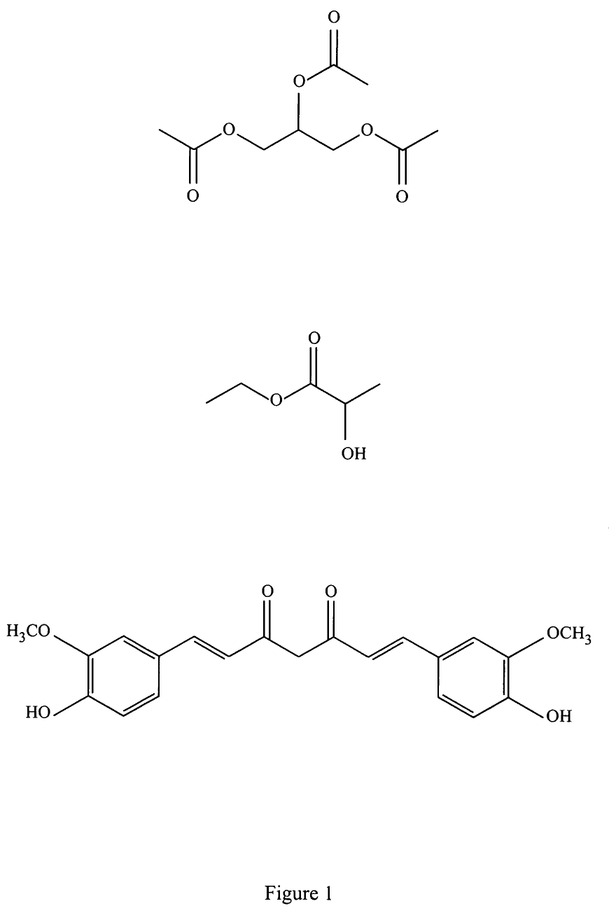

The following description of the preferred embodiment(s) is merely exemplary in nature and is in no way intended to limit the invention, its application, or uses. The simulant compositions contain triacetin (TA), also known as glyceryl triacetate, with an amount of ethyl lactate (EL) that is selected to obtain specific properties, e.g., vapor concentration, vapor pressure, volatility, apparent molecular weight, persistence, viscosity, solubility, response to oxidative and perhydrolysis decontaminants, etc, to correspond to a specific chemical toxant, e.g., the chemical warfare agents, methylphosphonothioic acid (VX), sulfur mustard (HD), and Tabun (GA), Sarin (GB), and Soman (GD), the G-agents. The chemical structures of the ingredients are shown in FIG. 1.

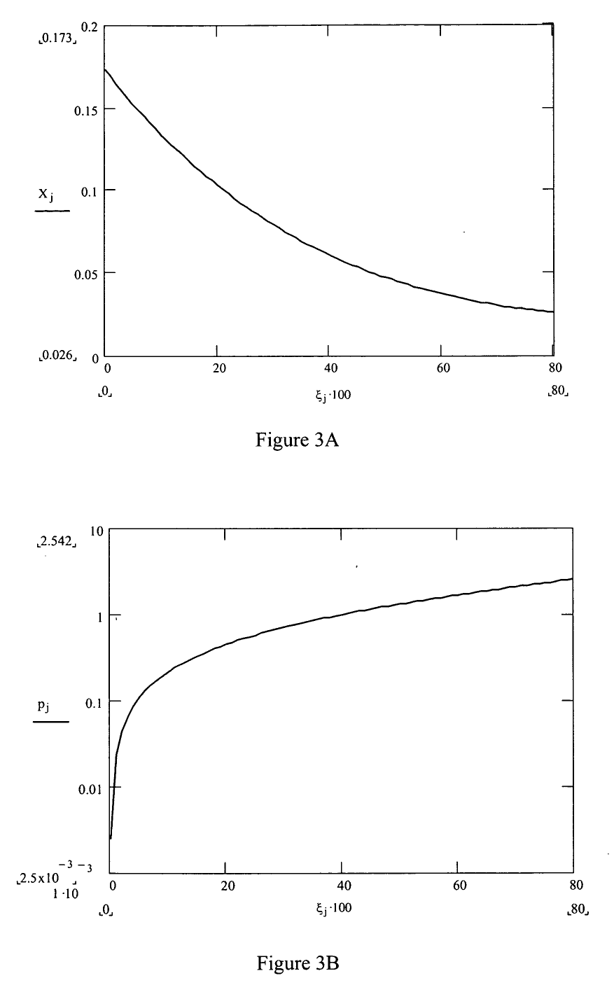

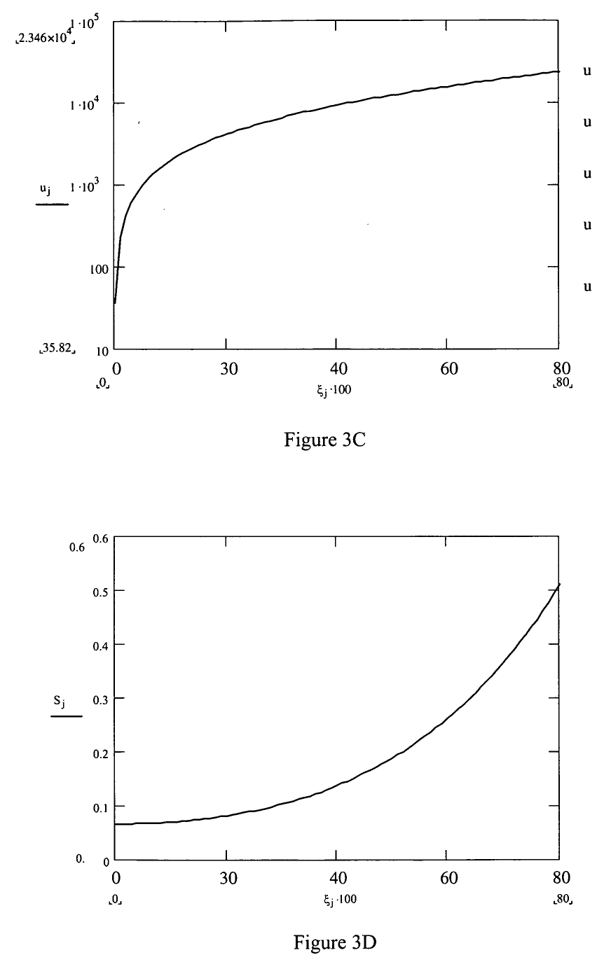

TA principally comprises the simulant for VX. The simulant group based on TA and containing various amounts of EL to obtain agent specific characteristics for the G-series (G-family) and sulfur mustard and related H-series (H-family) agents exhibits excellent detectability by photo-ionization vapor detectors (PID) (FIG. 3). The EL is a safe GRAS food grade component that is miscible in water. TA, which comprises the base of the simulant mixtures, has modest solubility in water.

The selection of TA and EL as the principal ingredients is also made because they have good material compatibility with most commonly found materials and do not dissolve or damage synthetic textiles that are commonly used to make garments. Another characteristic of these ingredients is their relative persistency (relative to water), RPr. At room temperature, for TA, RPr=2030, and for EL, RPr=5.7. So, compositions comprising mixtures of TA and EL can provide values of RPr ranging between the limiting RPr of the two ingredients. Further, TA has a relatively high boiling point temperature, namely T_{b}=258° C., and low volatility, so that in the composition, it does not act as a solvent that can be evaporated to leave behind the EL (T_{b}=154° C.), which has a much lower viscosity (EL: 2.63 cSt at 20° C. vs TA: 14.7 cSt at 25° C.). Thus, the viscosity and the RPr of the composition increases as the EL slowly evaporates from the composition. As TA is less soluble in water than EL, the solubility of the simulant compositions decreases as the EL evaporates and the simulant compositions “weather”.

A small admixture of curcumin or other non-toxic fluorescent taggant is included in the composition so that the simulants are visible upon illumination by UV light. The TA ester base family of chemical simulants without an added fluor exhibits no significant background fluorescence with an excitation wavelength in the near UV or visible violet up to a wavelength of 450 nm.

There are many exemplary non-toxic fluorescent taggants, which include optical brighteners and whiteners used in cosmetics and in textiles, as described in Berthiaume et al (U.S. Pat. No. 5,830,446), Cohen (U.S. Pat. No. 6,313,181), and Charbit (US 2005/0137117), and prior art cited in these references. Such fluorescent compounds are available from Ciba-Geigy under the tradename Tinopal®. Others are offered by BASF under the tradename Calcofluor®.

In a preferred embodiment, curcumin is selected as the taggant because of its fluorescence yield with excitation in the 355-460 nm wavelength range and an emission curve with a broad maximum in the vicinity of about 470-530 nm. With UV excitation, i.e., wavelength <400 nm, the emission can be seen in normal indoor lighting conditions, in moderate to low sunlight conditions, and at night.

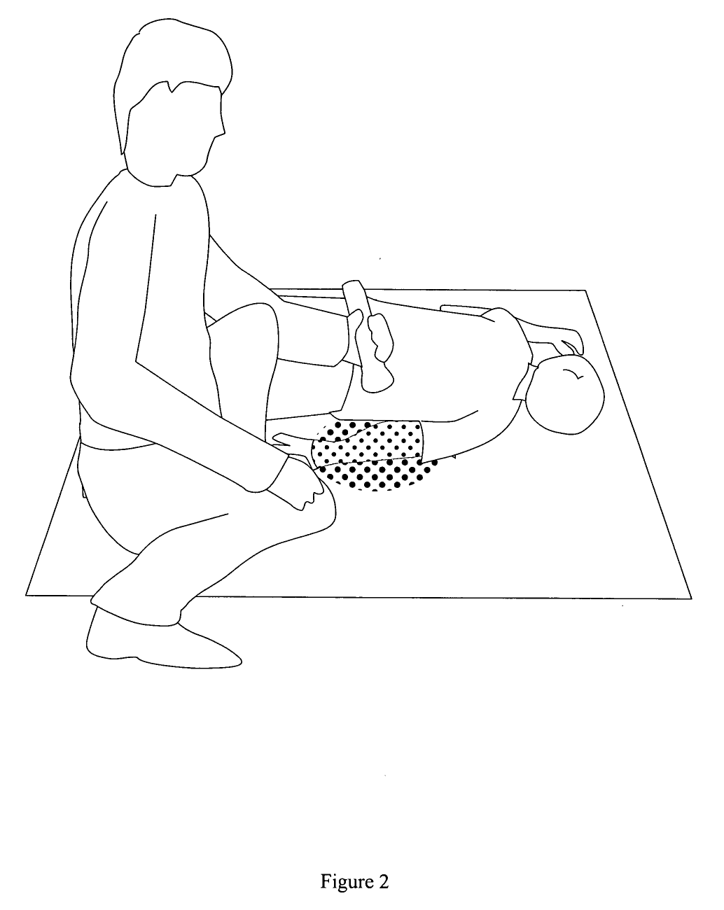

The ingredients shown in FIG. 1 (see Table I) are selected as GRAS, are listed in the International Dictionary of Cosmetics, and are non-irritating to human skin and mucous membranes, which is in contrast to simulants based on salicylate esters such as methyl salicylate. The compositions are safe for use in unrestricted areas by individuals in training. In a preferred embodiment, the taggant is curcumin, a product of ethanol extraction of the spice Turmeric. Information pertaining to curcumin is shown in Table II. When applied to a person, garments, equipment, or other objects, the simulant can be viewed when illuminated by a UV light, as shown in FIG. 2.

TABLE I
Simulant Ingredients

| | Empirical | | |
|---|---|---|---|
| Ingredient | Formula | Other Names | Trade Names |
| Ethyl Lactate | C₅H₁₀O₃ | 2-hydroxypropanoic | AEC Ethyl Lactate (A&E |
| (CAS# 97-64-3) | | acid; ethyl ester lactic | Connock); CPS Ethyl Lactate |
| | | acid; ethyl ester | (CPS); Ethyl Lactate FCC (Purac); |
| Triacetin | C₉H₁₄O₆ | acetic, 1,2,3- | Eastman Triacetin (Eastman |
| (CAS# 102-76-1) | | propanetriyl ester; | Chemical); PRIACETIN 1580 |
| | | glyceryl triacetate; | (Uniqema Europe); Unitolate GTA |
| | | 1,2,3-propanetriol | (Universal Preserv-A-Chem) |
| | | Triacetate; (tri-) acetin | |
| curcumin | C₂₁H₂₀O₆ | 1,7-bis(4-hydroxy-3- | curcumin C3 Complex (Sabinsa) |
| (CAS# 458-37-7) | | methoxyphenyl)hepta 1, | |
| | | 6-diene-3,5-dione; CI | |
| | | 75300; 1,6-heptadiene- | |
| | | 3,5-dione; 1,7-bis(4- | |
| | | hydroxy-3- | |
| | | methoxyphenyl)-; | |
| | | (E,E)_; Tumeric | |

TABLE II
CURCUMIN Information

| | |
|---|---|
| SOURCE | Derived from turmeric by extraction |
| | with ethanol |
| APPEARANCE | Orange-yellow, crystalline powder; |
| | insoluble in water |
| NOMENCLATURE | Turmeric yellow; diferuloylmethane; |
| | (E,E)-1,7-bis(4-hydroxy-3- |
| | methoxyphenyl)-1,6-heptadiene-3,5 |
| | dione |
| FORMULA | C₂₁H₂₀O₆ |
| MOLECULAR WEIGHT | 368.39 daltons |

The compositions of simulants for the chemical warfare agents GB, HD, and VX are shown in Table III. The simulant for VX comprises TA plus the fluorescent taggant. The simulant for HD comprises about 5% EL and about 95% TA plus the fluorescent taggant. The designation of the GB simulant as G-s is made as a generic choice for the G-series or G-family of agents. As described below, the proportions of TA and EL can also be selected to simulate a specific agent within the G-series, such as GA and GD. Compositions with as much as 70% to 80% EL by weight provide vapor, volatility, and viscosity characteristics that are easily differentiated according to the amount of TA and EL. For compositions with fraction of EL greater than about 80%, these characteristics do not vary much with fraction of EL in the composition. However, solubility does continue to increase as the amount of EL is increased.

FIGS. 3A-3D shows the characteristics of the TA-based simulants as functions of the EL percentage by weight of the composition, FIG. 3A viscosity (St); FIG. 3B vapor pressure (Torr); FIG. 3C volatility (mg/m³); and FIG. 3D solubility (g/g). These characteristics have been measured and the curves shown in FIGS. 3A-3D are based on the measured data and on simple composition rules. For example, the viscosity (ν) of the composition is given by the Grunberg equation:

ln ν=χ₁ ln νν₁+χ₂ ln ν₂+χ₁χ₂G

where G=−2.8 for the TA and EL mixtures, and χ₁ and χ₂ are the mole fractions and ν₁ and ν₂ are the viscosities of the two components. The vapor pressure is the sum of the partial pressures, which are given by Raoult's law. The volatility is calculated from the partial pressures. The solubility S is estimated as a fit of the following equation to the measured values given in Table IV:

S=χ₁²S₁+χ₂²S₂+gχ₁χ₂(S₁+S₂),

where g=0.15, and S₁ and S₂ are the solubilities of the two components. This equation is most accurate for small and large fractions of EL and overestimates the solubility by up to 25% for compositions with fraction of EL by weight in the vicinity of 50%.

The corresponding physical properties for the formulations of Table III are shown in Table IV. For comparison, the physical properties of the chemical agents and exemplary simulant compositions are shown in Table IV. The V-s simulant being a composition of TA and the fluorescent taggant has characteristics that are predominantly those of TA.

In the comparison in Table IV, it is seen that the solubility and viscosity of the H-simulant are significantly greater than for HD. While this may be viewed as a defect in the fidelity of the H-simulant, it is of practical advantage because the greater solubility in water reduces the amount of water necessary for clean-up after a training exercise. The greater viscosity does make removal of the simulant slightly more difficult than removal of the agent, but with the lower surface tension of the simulant, the sorption characteristics are similar.

TABLE III
Exemplary Simulant Compositions for
G, H, and V chemical agent toxants.

| Type | Triacetin (%) | Ethyl Lactate (%) | curcumin (%) |
|---|---|---|---|
| G-s | 48-52 | 48-53 | 0.005-0.010 |
| H-s | 94-96 | 4-6 | 0.005-0.010 |
| V-s | 98-99+ | — | 0.005-0.010 |

The vapor concentration associated with a liquid pool or film of simulant depends on the relative proportions of TA and EL. Because TA has a low vapor pressure (Pᵥ=0.0025 Torr @ 25° C.), a vapor density of 7.52 relative to air, and high molecular weight (218.2), it is a good simulant for the volatility and vapor concentration of VX, which also has a very low vapor pressure. In contrast, EL has a much greater vapor pressure (Pᵥ=5 Torr @ 30° C.), a vapor density of 4.07, and a molecular weight of 118.1. Consequently, a 50-50% mixture of TA and EL is a good simulant for the vapor properties of GB.

TABLE IV
Chemical Simulant and Toxant Physical Properties

| PROPERTY | EL | G-s | H-s | V-s |
|---|---|---|---|---|
| Apparent | 118.1 | 153.3 | 212.7 | 218.2 |
| Molecular Weight | | | | |
| (amu) | | | | |
| Density (g/cm³) | 1.03 | 1.102 | 1.159 | 1.163 |
| Kinematic | 2.63 | 4.65 | 14.45 | 16.74 |
| Viscosity (cSt) | | | | |
| Vapor Pressure | 3.7 | 1.1 | 0.063 | 0.0035 |
| (Torr @ 25° C.) | | | | |
| Surface Tension | 30.0 | 29.2 | 32.8 | 33.2 |
| (dynes/cm) | | | | |
| Solubility (ml/100 | Miscible | 15 | 6.7 | 6.5 |
| ml of DIW) | | | | |
| Volatility | 17130 | 6034 | 493 | 17.9 |
| (mg/m³) | | | | |
| Persistency | 5.7 | 8.7 | 108 | 2030 |

| PROPERTY | GA | GB | GD | HD | VX |
|---|---|---|---|---|---|
| Apparent | 162.1 | 140.1 | 182.2 | 159.07 | 267.4 |
| Molecular | | | | | |
| Weight (amu) | | | | | |
| Density (g/cm³) | 1.07 | 1.102 | 1.02 | 1.27 | 1.01 |
| Kinematic | 2.18 | 1.28 | 3.10 | 3.52 | 12.26 |
| Viscosity (cSt) | | | | | |
| Vapor Pressure | 0.057 | 2.9 | 0.4 | 0.106 | 0.0007 |
| (Torr @ 25° C.) | | | | | |
| Surface Tension | Not | 26.5(20° C.) | 24.5(26.5° C.) | 43.2(20° C.) | 32.0(20° C.) |
| (dynes/cm) | Found | | | | |
| Solubility | 9.8(25° C.) | 100 | 2.1(25° C.) | 0.09(22° C.) | 3(20° C.); |
| (ml/100 ml of DIW) | | (miscible) | | | miscible |
| | | | | | (<9.4° C.) |
| Volatility | 490 | 22000 | 3900 | 906 | 10.5 |
| (mg/m³) | | | | | |
| Persistency | 200 | 3 | 10 | 80 | 6000 |

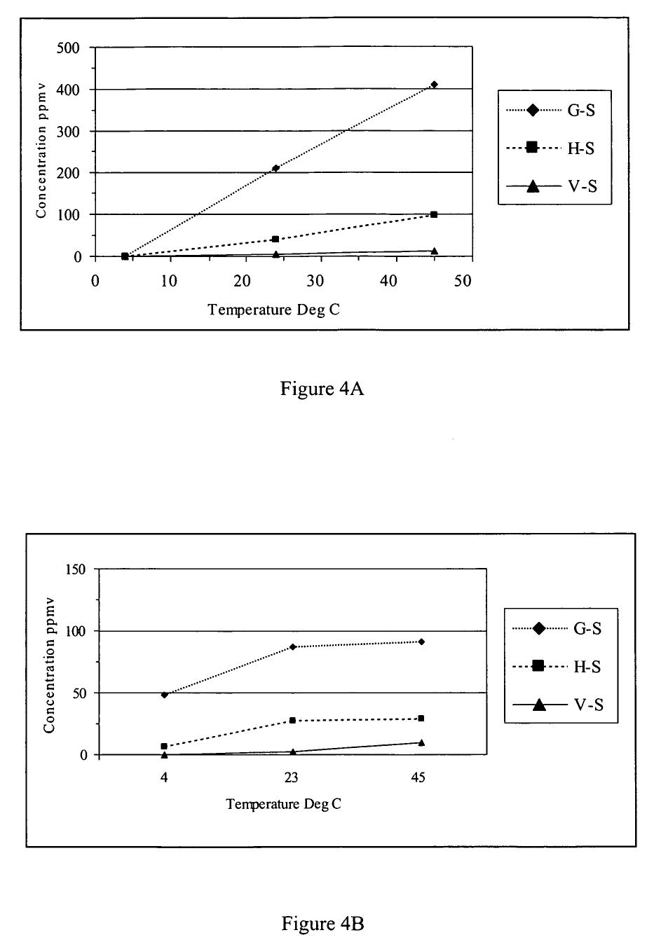

The temperature dependence of the vapor pressures of the chemical simulants is shown in Table V. The actual vapor pressure of the chemical simulants was determined by laboratory analysis and also measured using the MiniRae 2000™ photo-ionization detector (PID) instrument. Laboratory vapor pressure and concentration volatility data are reported in Table VI. The data are presented in FIGS. 4A and 4B.

TABLE V
Determination of Simulant Vapor Concentration
at Three Temperatures as Measured by a PID.

| | | VAPOR | | VAPOR |
|---|---|---|---|---|
| | | CONCENTRATION | | PRESSURE |
| SIMULANT | TEMP (° C.) | mg/m³ | ppmv | (mm Hg) |
| G-s | 4 | 2287 | 453 | 0.34 |
| | 24 | 8265 | 1290 | 1.00 |
| | 45 | 10395 | 1652 | 1.26 |
| H-s | 4 | 1675 | 192 | 000 |
| | 24 | 1361 | 156 | 0.120 |
| | 45 | 2776 | 319 | 0.240 |
| V-s | 4 | 000 | 000 | 000 |
| | 24 | 421 | 47 | 0.036 |
| | 45 | 1810 | 202 | 0.150 |

TABLE VI
Vapor Characteristics of Chemical Simulants Detectable by PID

| | | |
|---|---|---|
| | Vapor | |
| Molecular | Pressure (mm | Volatility |

| Simulant | Weight | Hg @ 20° C.) | (mg/m³) | (ppmv) |
|---|---|---|---|---|
| G-s | 153.37* | 1.10 | 8284 | 1318 |
| H-s | 212.76* | 0.063 | 723 | 83 |
| V-s | 218.20 | 0.0035 | 34.8 | 3.9 |

*apparent molecular weight

The test data show that the ppbRae™ PID vapor detector response is saturated at vapor concentrations greater than 100 ppmv. However, a more recent model of the ppbRae™ has an increased dynamic range as seen in FIG. 3 (upper).

The fluorescence of the simulants is optimized so that the maximum signal is obtained for all simulants in response to illumination by UV light in the UVA region of the spectrum. An example of a suitable UV light source is a light-emitting diode (LED) flashlight with narrowband emission of the order of one Watt, e.g., 0.3 to 5 W, at a wavelength of approximately 390 nm. Broadband UV sources are also suitable.

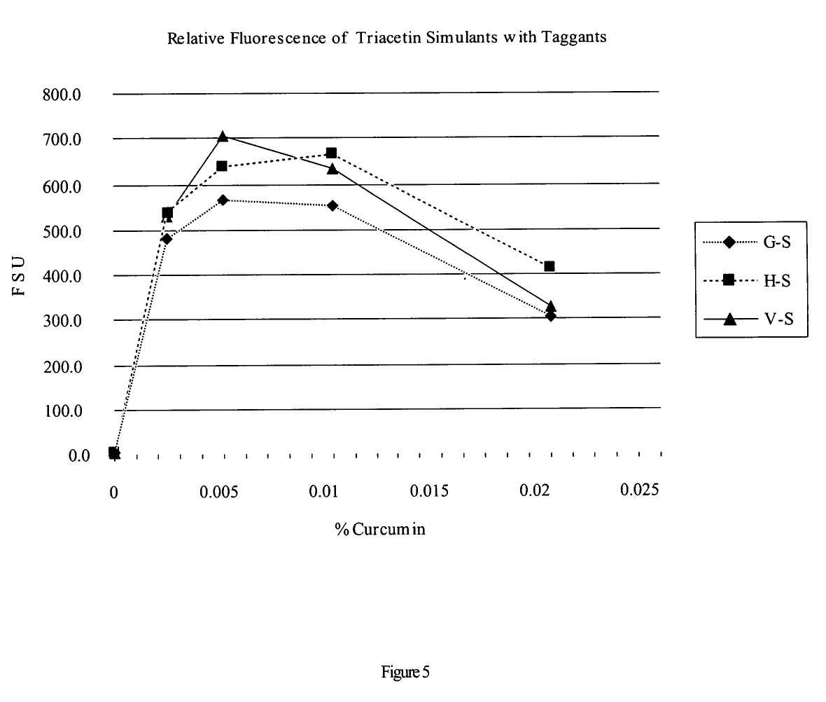

The fluorescent emission of TA ester-based simulants with curcumin fluorescent taggant exhibits a broadly peaked curve as a function of curcumin concentration. This response curve is seen in FIG. 5. Fluorescence is observed for curcumin concentration in the range 0.0001% to about 0.04%. Strong fluorescent response is observed for curcumin concentration in the range 0.001% to 0.025%. For concentration greater than approximately 0.01%, quenching effects reduce the fluorescent response. In a preferred embodiment, the curcumin concentration is in a range 0.002% to about 0.015%. In a more preferred embodiment, the curcumin concentration is between 0.005% and 0.010%. Commercially, the curcumin concentration is between 0.001% and 0.05%

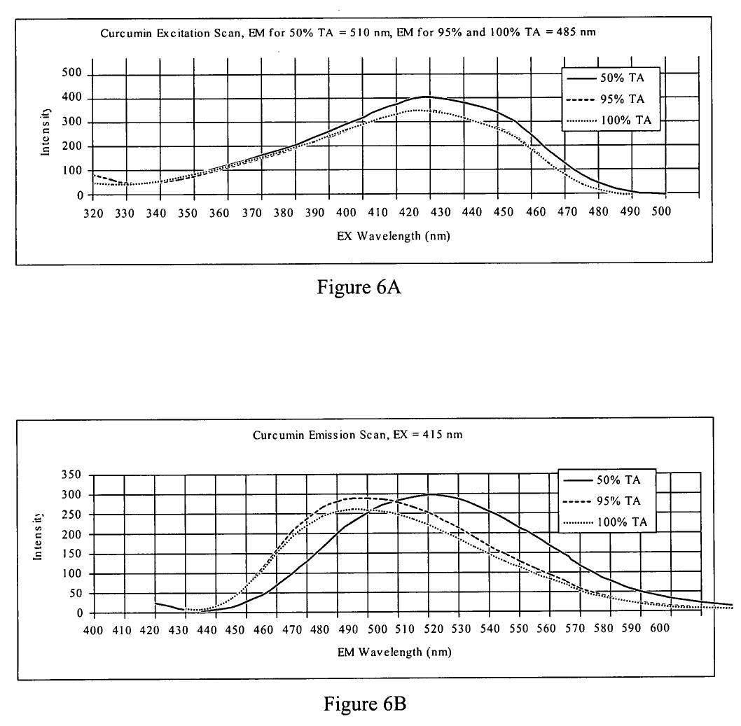

The excitation spectra and emission spectra of the simulant compositions were measured in laboratory tests. FIGS. 6A and 6B shows that the broad excitation maxima for all three simulants with taggant have peaks that are close to 415 nm, which indicates that all of the simulants can be visualized by using a single light source. FIG. 6B shows that as EL concentration increases, the curcumin emission wavelength is red shifted to a longer wavelength. However, the effect is small and the emission remains in the visible range.

Preparation of the compositions by mixing of the ingredients is straightforward. As TA and EL are both readily mixed together, the order of addition is not important. Because the curcumin can be readily mixed with either ingredient, for example by stirring powdered curcumin into a batch of TA or TA plus EL or EL, it is found that addition of the curcumin into the TA or after mixing TA and EL is best to obtain the desired concentration of curcumin in the composition.

For small batches of simulant, the addition of small amounts of fluor to obtain the desired concentration is made practical by first mixing the fluor in a solvent or carrier to make a solution or colloidal suspension on suitable concentration in the range of 0.5% to 25%, more preferably in the range 1-10%, so that an easily measured quantity can be added to the simulant batch. However, it is not desirable that the solvent remain in the final simulant as the solvent may lead to undesirable incompatibility of the simulant with plastics and textiles, and so, lead to adverse effects on garments and objects during the use of the simulant. The solvent may also contribute a vapor component that may interfere with training, sampling, detection, or the evaluation of decontamination effectiveness. To avoid the residual presence of such a solvent and to ensure that the final composition is GRAS and non-toxic, a suitable solvent is one with low boiling temperature in comparison with the boiling temperatures of TA (258° C.) and the EL (˜151-155° C.) and also with their flash point temperatures so that the solvent can be removed from the composition by evaporation. One example is acetone, which, at standard pressure, has a boiling point of 56° C. Thus, the acetone can be evaporated safely by gentle warming of the composition at approximately 60° C., which is well below the flash point temperatures of TA and EL vapors and the boiling point temperatures of the TA and EL, so that there is no significant loss or decomposition of these ingredients from the composition.

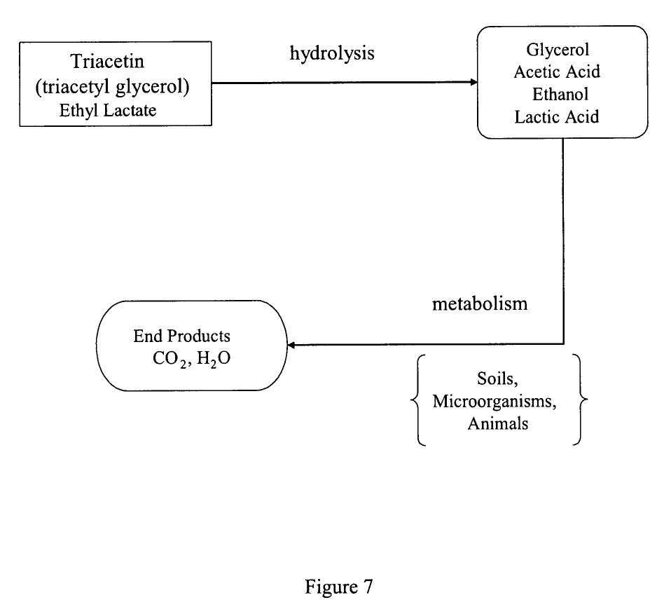

The ingredients of the simulants are all bio-degradable and will not generate any hazardous waste byproducts. Biodegradation pathways of the TA and EL ingredients of the chemical simulants are shown in FIG. 7. The end products are environmentally safe. The simulants respond well to cleanup with decontaminants or with hot soapy water. The alkaline chemistry of the soap solutions will affect partial chemical hydrolysis of the esters converting them into water soluble products easily removable by washing with water alone. Clean up of simulant spills and disposal of excess chemical simulant can easily be neutralized by addition of alkaline soap and water or bicarbonate of soda (baking soda).

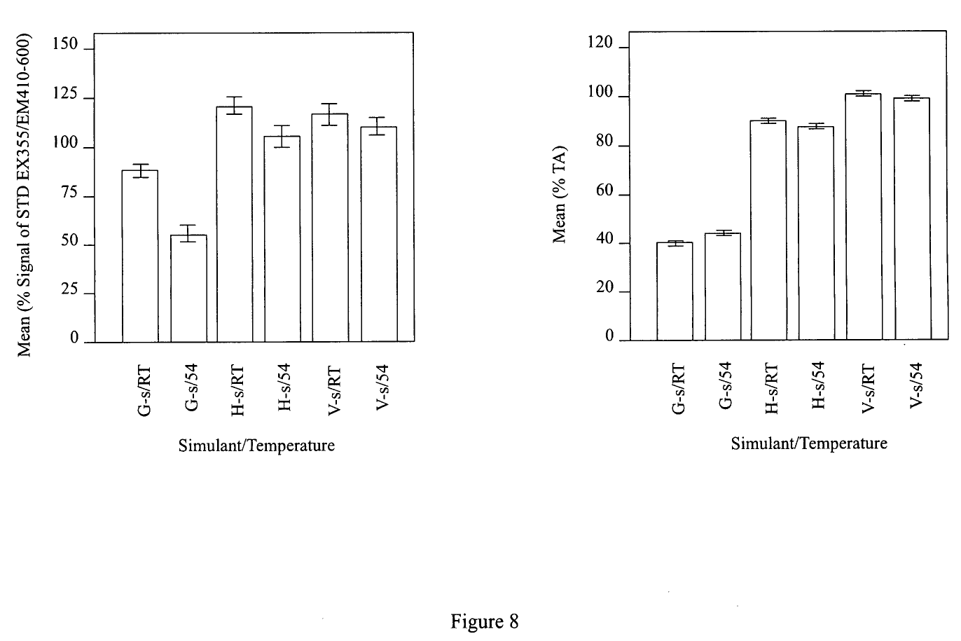

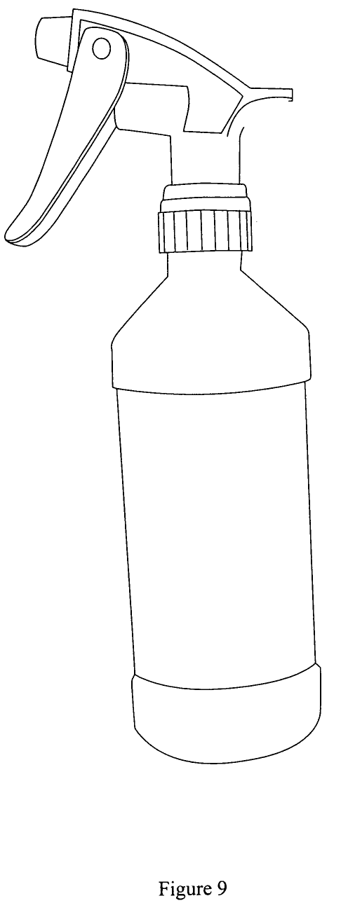

The shelf life of the compositions, i.e., the storage stability of the fluorescent taggant in the simulant formulations, was measured by accelerated shelf life testing at an elevated temperature (FIG. 8A, FIG. 8B). By Arhennius scaling, five months of testing at approximately 54° C. corresponds to aging at room temperature for more than three years. The chemical simulants were divided into small aliquots and put into sealed vials, which were placed in a temperature-controlled environment. Samples were removed on a monthly basis for chemical assay. The chemical stability of the TA and EL ester components was determined by HPLC assay with separation on an RP C18 column using a methanol:water (40:60) mobile phase. The chemical stability of the curcumin was measured using fluorescence measurements performed on a Turner N700 Filter Fluorometer and the Victor³ Microplate Reader. After five months, the V-s and H-s show no significant loss of fluorescence at room temperature (RT) or at elevated temperature (FIG. 8A). For G-s, there is ˜12% loss of fluorescence in the sample stored at room temperature and a 40% loss at elevated temperature. The chemical stability of the TA in the same aged samples was measured by HPLC (FIG. 8B). After five months, there was no significant loss of TA at either temperature for any of the three simulants. The data indicate the EL is also stable (data not shown). Thus, it is concluded that the simulants will have acceptable fluorescent activity after 3 years of storage at room temperature.

In vitro eye and skin sensitivity testing of the V-s and G-s formulations were performed. For the sake of cost, the H-s was not tested because its effect is expected to be intermediate between V-s and G-s. Skin and eye sensitivity testing was performed by Batts Laboratories using the Ocular and Dermal Irritection® assays. These assays are standardized and quantitative in vitro ocular and dermal irritation tests, which utilize changes of relevant macromolecules to predict non-irritancy to acute ocular and dermal irritancy of chemicals and chemical formulations. Table VII shows the test results and predicted irritancy effect on eyes and skin. However, all chemical components of the simulant group, upon percutaneous adsorption into skin are hydrolyzed by esterase enzymes into harmless biochemicals such as acetic acid, lactic acid and glycerol. Thus, in practice, no irritation is expected from skin exposure, and no irritation in fact was experienced by applying the simulant group on human skin.

TABLE VII
Summary of Eye and Skin Sensitivity Testing

| | | Ocular Irritection ® | | Dermal Irritection ® | |
|---|---|---|---|---|---|
| | | Results | | Results | |
| | | | Predicted | | Predicted Dermal |
| Simu- | Dose | IDE^{a} | Ocular | HIE^{b} | Irritancy |
| lant | (μl) | Score | Irritancy | Score | Classification |
| V-s | 25 | 16.8 | Mild | 0.68 | Non-Irritant |
| | 50 | 18.4 | | 0.91 | Non-Irritant/ |
| | 75 | 20.7 | | 1.01 | Irritant |
| | 100 | 22.9 | | 1.16 | |
| | 125 | 23.8^{c} | | 1.27^{c} | Irritant |
| G-s | 25 | 17.0 | Mild | 1.03 | Non-Irritant/ |
| | 50 | 18.8 | | 1.10 | Irritant |
| | 75 | 25.6 | | 1.69 | Irritant |
| | 100 | 28.4 | Mild/ | 1.98 | |
| | 125 | 31.0^{c} | Moderate | 2.17^{c} | |

^{a}Irritection ® Draize Equivalency: 0.0-12.5, minimal; 12.5-30.0, mild; 30.0-51.0, moderate; 51.0-80.0, severe
^{b}Human Irritancy Equivalent: 0.00-0.90, non-irritant; 0-90-1.20, non-irritant/irritant; 1.20-5.00, irritant
^{c}Maximum Qualified Score

An intended use of the simulants is a training aid in the areas of detection, contamination avoidance, contamination containment, decontamination, or contaminated material removal and handling. The simulants can be used under a broad range of ambient temperature (e.g., 0-50° C.) and weather conditions.

All of the simulants were characterized quantitatively for their performance on a variety of surfaces, including common building materials, PPE, civilian clothing, and skin (porcine and human) in the presence and absence of interferents. Results of the laboratory testing described here show that the simulants will perform well in the field and are safe to use on human skin and mucous membranes, and that they will continue to perform in the presence of common interferents, such as skin lotion, perspiration, paint, motor oil and dust. The simulants are GRAS according to the International Dictionary of Cosmetic Ingredients.

Simulant performance was measured on 15 various materials as well as on porcine skin samples and human volunteers. Additionally, various common compounds that are anticipated to be present likely during training exercises were evaluated for their potential to interfere with fluorescent signals. These materials include porous and non-porous materials. The non-porous test materials included the polymers and fabric of which PPE are made, glass, plastics, and metal. Porous test materials included construction materials such as concrete, wood, drywall, and floor tile, porcine skin, and clothing materials.

In a typical test, the simulant is applied as an aerosol spray. The fluorescent signal was measured with a UVX-300 Illuminator instrument. Visual observation was made. The visible fluorescence of the UV excited simulants was readily observed on the non-porous materials. For flat, non-porous surfaces, the simulant either forms a thin film coating or forms droplets and small puddles, as the behavior depends on the wet-ability of the surface. In contrast, for porous materials, such as unfinished wood and porous construction materials, the simulant is partially absorbed. In this case, the fluorescent signal is less and may be more diffuse, but it is still visible, generally. In the case of fabrics that contain whiteners, e.g., previously detergent laundered cotton and cotton blend garments, the fluorescence of the simulant may appear either as a low additional fluorescence to a low background fluorescence of the fabric without the simulant, or it may appear as a quenched area on fabric that has high background fluorescence. In either case, the fabric fluorescence is visibly altered by the presence of the simulant. On porous materials that are highly absorbent, such as soils, concrete, or carpet, the amount of simulant that remains on the surface may be very small, and so, the fluorescent signal may also be small. For such materials, detection is best done with a vapor detecting instrument.

Observation of the fluorescence of simulant applied on porcine skin and human skin (Caucasian and African-American) showed that the simulants are visible both with and without UV illumination as the curcumin can be excited by violet light with wavelength in the range 400-470 nm. However, in normal room lighting and sunlight, the intensity of such violet light is low, and so the visible fluorescence is low. In contrast, illumination with a UV emitting LED flashlight or other UV lamp with suitable spectral content provides substantially brighter fluorescence that is readily observed.

The fluorescence is also readily observed when the surface is contaminated with commonly occurring interferents, although the appearance is different than for an otherwise clean surface. These interferents included skin lotions, perspiration, water, motor oil, and dust/dirt. In contrast, the observation of the fluorescence is made difficult by bright ambient light (e.g., sunlight), paints and other materials containing whiteners or other highly fluorescent compounds, and highly reflective surfaces.

Chemical toxants may be encountered on surfaces as the result of a point release such as by a sprayer or an explosive dissemination or dispersion device, or as deposition from an aerosol cloud, or as condensation of a toxant vapor, or as spatter from other spraying, or as contamination spread by contact with other contaminated surfaces. As a result, toxant contamination may comprise streaks (e.g., >0.5 mm thickness), large droplets (5-500 μl), fine droplets (<5 μl), a thin film, or puddles or pools, or combinations thereof. The simulants may be applied by similar means to those anticipated for an attack or other contamination event. The simulants may also be applied by pouring, spraying, brushing, wiping, or smearing. One preferred applicator is a handheld spray bottle as shown in FIG. 9. Such a sprayer is a flexible 16 oz., high density polyethylene (HDPE) dispensing bottle. The sprayer may be replaced by a pour spout or the simulant may be poured from such a bottle onto a surface, or poured into a cap, spoon, or wipe for application to a surface. By such means, the simulant may be applied to surfaces in an amount that may be typical of anticipated toxant threats, e.g., at challenge levels of 0.1 to 10 g/m².

The use of the simulants for chemical toxants may be made by the method comprising the following steps:

1. Designation of a scene or place and the objects and persons therein that are to be exposed to the simulant;

2. Application of the simulant by aerosol spraying, pouring, brushing, sponging, wiping, or other appropriate means of dissemination so that the simulant contacts the surfaces of objects such as walls, persons, equipment, etc, or so that the simulant forms an aerosol cloud with droplets that remain suspended for sufficient time for sampling, detection, or decontamination;

3. Detection of the contamination (represented by the simulant) by visual inspection of the fluorescence of the simulant when illuminated by UV light with wavelength in the 355-400 nm range and/or the detection of the simulant by a chemical toxant detector, e.g., a photo-ionization detector (PID) and/or sampling of the contamination (represented by the simulant) for subsequent analysis or detection;

4. Optionally, the conduct of decontamination means, which may include physical removal of contamination or contaminated articles, the application of decontaminants, or other means to remove or neutralize the contamination (represented by the simulant);

5. Optionally, the evaluation or scoring or the effectiveness of the training, sampling, decontamination, research, or success of the activity using the simulant for another purpose; and

6. The cleaning up of the scene, place, objects, persons, etc by use of appropriate means, for example, water, soap and water, or decontaminant. It should be obvious to those practiced in the art that the above descriptions are exemplary and not meant to be limiting as the method for use, the specific compositions, and the choice of fluorescent taggant may have many variations.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A simulant for a chemical toxant comprising:
glyceryl triacetate.

2. The simulant for a chemical toxant of claim 1, further comprising curcumin as the fluorescent taggant.

3. The simulant for a chemical toxant of claim 2, wherein the curcumin comprises between 0.001% and 0.05% of said simulant.

4. The simulant for a chemical toxant of claim 1 wherein said simulant has one or more properties to correspond to a specific agent, said properties being selected from the group consisting of: vapor pressure, vapor concentration, volatility, apparent molecular weight, persistence, viscosity, solubility, response to oxidative, hydrolysis, and perhydrolysis decontaminants.

5. The use of the simulant for a chemical toxant of claim 2, wherein the simulant is applied to the skin of a person or animal.

6. A simulant for a chemical toxant comprising:
glyceryl triacetate; and
a selected amount of ethyl lactate so that a simulant composition containing the said selected amount of ethyl lactate has one or more properties that correspond to a specific chemical toxant or a family of chemical toxant.

7. The simulant for a chemical toxant of claim 6, wherein said simulant contains between more than 0% and 80% ethyl lactate.

8. The simulant for a chemical toxant of claim 6, further comprising curcumin as the fluorescent taggant.

9. The simulant for a chemical toxant of claim 8, wherein the curcumin comprises between 0.001% and 0.05% of said simulant.

10. The simulant for a chemical toxant of claim 6, wherein said simulant has one or more properties to correspond to a specific agent, said properties being selected from the group consisting of: vapor pressure, vapor concentration, volatility, apparent molecular weight, persistence, viscosity, solubility, response to oxidative, hydrolysis, and perhydrolysis decontaminants.

11. The use of the simulant for a chemical toxant of claim 8, wherein the simulant is applied to the skin of a person or animal.

12. A set of simulant compositions for chemical toxants, comprising:
triacefin;
a generally regarded as safe (GRAS) fluor;
a selected amount of ethyl lactate so that a simulant composition containing the said selected amount of ethyl lactate has one or more properties that correspond to a specific chemical toxant or family of chemical toxants.

13. The use of the set of simulant compositions for chemical toxants of claim 12, where said simulant is applied to the skin of a person or animal.

14. The set of simulant compositions for chemical toxants of claim 12, wherein said GRAS fluor is curcumin.

15. The set of simulant compositions for chemical toxants of claim 12, wherein said selected amount of ethyl lactate is between more than 0% and about 80%.

16. The set of simulant compositions for chemical toxants of claim 12, wherein the said one or more properties that correspond to specific agents are selected from the group consisting of: vapor pressure, vapor concentration, volatility, apparent molecular weight, persistence, viscosity, solubility, response to oxidative and perhydrolysis decontaminants.

17. The use of a simulant composition for chemical toxants comprising:
triacetin;
a GRAS fluorescent taggant;
for one or more purpose selected from the group consisting of: training in the use of chemical toxant detectors, training in the decontamination of persons, equipment, or places that are contaminated with chemical toxants, conducting research, evaluating training or decontamination effectiveness, and the study of the transport and spread of chemical toxants.

18. The use of a simulant composition for chemical toxants comprising:
triacetin;
an amount of ethyl lactate that is between more than 0% and 80% of the composition by weight;
a GRAS fluorescent taggant;
for one or more purpose selected from the group consisting of: training in the use of chemical toxant detectors, training in the decontamination of persons, equipment, or places that are contaminated with chemical toxants, conducting research, evaluating training or decontamination effectiveness, and the study of the transport and spread of chemical toxants.

19. The method of training in the use of chemical toxant detection equipment, sampling methods, or decontamination procedures and technologies, or conducting research in the transport phenomena of chemical toxants, and research and development of countermeasures for chemical toxants, the method comprising:
using simulant compositions for chemical toxants comprising triacetin, and a GRAS fluorescent taggant;
by said simulant composition, the application on and contacting of one or more of the group comprising inanimate objects, plants, persons, and animals; and
observing and/or detecting the simulant on the surface of an object, person, plant, or animal, either by visual observation, by detection with detecting equipment, or by visual observation of the fluorescence of the simulant.

20. The method of training in the use of chemical toxant detection equipment, sampling methods, or decontamination procedures and technologies, or conducting research in the transport phenomena of chemical toxants, and research and development of countermeasures for chemical toxants according to claim 19, further comprising:
performing sampling, decontamination, research in the transport phenomena of chemical toxants, or research and development of countermeasures for chemical toxants.

21. The method of training in the use of chemical toxant detection equipment, sampling methods, or decontamination procedures and technologies, or conducting research in the transport phenomena of chemical toxants, and research and development of countermeasures for chemical toxants, the method comprising:
using simulant compositions for chemical toxants comprising triacetin, an amount of ethyl lactate that is between more than 0% and 80% of the composition by weight, and a GRAS fluorescent taggant;
by said simulant compositions, the application on and contacting of one or more of the group comprising inanimate objects, plants, persons, and animals; and
observing and/or detecting the simulant on the surface of an object, person, plant, or animal, either by visual observation, by detection with detecting equipment, or by visual observation of the fluorescence of the simulant.

22. The method of training in the use of chemical toxant detection equipment, sampling methods, or decontamination procedures and technologies, or conducting research in the transport phenomena of chemical toxants, and research and development of countermeasures for chemical toxants according to claim 21, further comprising:
performing sampling, decontamination, research in the transport phenomena of chemical toxants, or research and development of countermeasures for chemical toxants.

23. A simulant for a chemical toxants, comprising:
triacetin, a generally regarded as safe (GRAS) fluor;
a selected amount of ethyl lactate such that said simulant composition has one or more properties that correspond to a specific chemical toxant or a family of chemical toxants; and
wherein said fluor is added to the composition as an ingredient comprising between 0.1% and 25% of a solution that has a solvent with relatively low boiling point in comparison with the flash point temperatures and boiling point temperatures of triacetin or ethyl lactate, and after dispersion and mixing of the fluor solution, the simulant with dispersed fluor solution is warmed for a sufficient period so that the said solvent is evaporated from the simulant so that the remaining composition is effectively free of the solvent of the fluor solution.

24. A method of preparation of a simulant that comprises triacetin, a generally regarded as safe (GRAS) fluor, and a selected amount of ethyl lactate so that a simulant composition containing the said selected amount of ethyl lactate has one or more properties that correspond to a specific chemical toxant or a family of chemical toxant, the method comprising:
mixing of a composition of glyceryl triacetate and ethyl lactate with the desired fractions by weight of the two ingredients;
adding a fluor to said glyceryl triacetate-ethyl lactate composition as an ingredient comprising between 1% and 25% of a solution that has a solvent with relatively low boiling point in comparison with triacetin or ethyl lactate, and said solution being in an amount so that said fluor corresponds to a desired fraction of the glyceryl triacetate-ethyl lactate composition that is in the range of about 0.001% to 0.1%;
mixing and dispersing the said fluor solution throughout the glycezyl triacetate-ethyl lactate composition;
warming the simulant composition and dispersed fluor solution at a temperature well below the flash point temperatures and boiling point temperatures of glyceryl triacetate and ethyl lactate for a sufficient period so that the said solvent is evaporated from the composition to result in a simulant composition that is effectively free of the solvent of the fluor solution.

25. The method of preparation of claim 24, wherein said fluor comprises curcumin and said solvent is acetone, and wherein said curcumin comprises between 0.001% and 0.05% of the resulting simulant composition.

26. A simulant for a chemical toxants, comprising:
triacetin and a generally regarded as safe (GRAS) fluor;
wherein said fluor is added to the composition as an ingredient comprising between 0.1% and 25% of a solution that has a solvent with relatively low boiling point in comparison with the flash point temperature and boiling point temperature of triacetin, and after dispersion and mixing of the fluor solution, the simulant with dispersed fluor solution is warmed for a sufficient period so that the said solvent is evaporated from the simulant so that the remaining composition is effectively free of the solvent of the fluor solution.

27. A method of preparation of a simulant that comprises triacetin and a generally regarded as safe (GRAS) fluor, the method comprising:
preparing a fluor solution with said fluor comprising between 1% and 25% of the said solution and said solvent having a relatively low boiling point in comparison with the boiling point and the flash point temperatures of triacetin;
adding said fluor solution to a quantity of glyceryl triacetate, and said solution being in an amount so that said fluor corresponds to a desired fraction of the simulant composition that is in the range of about 0.001% to 0.1%;
mixing and dispersing the said fluor solution throughout the glyceryl triacetate composition;
warming the simulant composition and dispersed fluor solution at a temperature well below the flash point temperature and boiling point temperature of glyceryl triacetate for a sufficient period so that the said solvent is evaporated from the composition to result in a simulant composition that is effectively free of the solvent of the fluor solution.

28. The method of preparation of claim 27, wherein said fluor comprises curcumin and said solvent is acetone, and wherein said curcumin comprises between 0.001% and 0.05% of the resulting simulant composition.
